(19) United States
(12) Patent Application Publication
(10) Pub. No.: US 2019/0134133 A1
(43) Pub. Date: May 9, 2019

(54) COMPOSITION FOR REDUCING ANXIETY IN A COMPANION ANIMAL

(71) Applicant: WISYS TECHNOLOGY FOUNDATION, INC., Madison, WI (US)

(72) Inventors: Cassie Lynn Van Hoof, Racine, WI (US); Daryl Sauer, Trevor, WI (US)

(21) Appl. No.: 16/185,835

(22) Filed: Nov. 9, 2018

Related U.S. Application Data
(60) Provisional application No. 62/583,625, filed on Nov. 9, 2017.

(51) Int. Cl. A61K 36/53; A61P 25/22; A61K 36/35; A61K 36/82; A61K 36/22; A61K 36/88; A61K 36/185; A61K 47/12; A61K 47/44; A61K 47/46; A61K 9/00; C11D 3/00; C11D 3/382; A01K 15/02; A01K 1/035

(52) U.S. Cl. CPC A61K 36/53; A61K 2236/10; A61K 36/35; A61K 36/82; A61K 36/22; A61K 36/88; A61K 36/185; A61K 47/12; A61K 47/44; A61K 47/46; A61K 9/007; C11D 3/0005; C11D 3/382; A01K 15/025; A01K 15/024; A01K 1/035; A61K 2236/331; A61K 2236/30; A61P 25/22

(57) ABSTRACT

The present invention provides a composition for reducing anxiety in a companion animal, particularly a feline. Such a composition is based on an extract of an ingredient mixture including catnip, valerian root, black tea, thyme, mango and, optionally, one or more of banana and slippery elm, the composition further comprising lavender. Methods of manufacture and use are further described.

### CROSS-REFERENCE TO RELATED APPLICATIONS

This application claims the benefit of U.S. Provisional Application No. 62/583,625, filed Nov. 9, 2017, which is incorporated herein by reference in its entirety.

### STATEMENT AS TO RIGHTS TO INVENTIONS MADE UNDER FEDERALLY SPONSORED RESEARCH AND DEVELOPMENT

Not applicable.

### FIELD OF THE INVENTION

The present invention relates to companion animal care. More particularly, the present invention relates to a composition for reducing anxiety in a companion animal, particularly a feline.

### BACKGROUND OF THE INVENTION

While often subtle in showing their insecurities, feline pets do suffer environment-induced anxiety as exhibited by nervousness, uneasiness and apprehension. These are normal and to-be-expected responses under certain circumstances, but can become problematic if they are excessively-severe or occur at inappropriate times. Common situations that cause a cat anxiety include: separation from an owner; moving to a new home; and the addition of a new family member (spouse, child or pet). However, the symptoms of an anxious cat may be difficult to attribute to one particular cause. Mild symptoms of anxiety in cats include: hiding more than normal; greeting owners exuberantly when they return home; and closely-following owners from room to room. More noticeable symptoms of anxiety in cats include urinating or defecating outside of a litter box; vomiting; and compulsively grooming themselves. In extreme cases, compulsive grooming leads to bald patches which require veterinary attention. While feline diseases can also exhibit these symptoms, the underlying condition is frequently an anxiety disorder.

In cases where a cat's anxiety does not result in self-harm requiring veterinary care, and the animal is otherwise healthy, providing the animal with a distraction such as a special toy or access to a perch or window may be a sufficient remedy. Alternatively, the pet owner may treat the cat with various nutraceutical, herbal and homeopathic formulations designed to soothe anxious animals. More recent home care products include sprays, diffusers, and wipes containing a synthetic feline facial pheromone alleged to make cats feel calm and relaxed. However, despite available options and recent advances, there remains a long felt need in the area for effective and low-cost formulations for reducing anxiety in companion animals, particularly cats.

### SUMMARY OF THE INVENTION

The present invention is an all-natural solution to redirecting feline aggression and anxiety. The product is applicable in the home, in shelters, and in veterinary hospitals where feline aggression and anxiety may persist.

According to one aspect of the invention, a composition for reducing anxiety in a companion animal comprises an extract of an ingredient mixture including catnip, valerian root, black tea, thyme, mango and, optionally, one or more of banana and slippery elm, the composition further comprising lavender.

The composition may further comprise a natural preservative. The natural preservative may be citric acid, an essential oil, or an herb. The natural preservative may be vinegar.

The ingredient mixture may be extracted by steam distillation to yield the extract.

The ingredient mixture may be extracted by microwave assisted extraction (MAE) to yield the extract.

The ingredient mixture may be pre-mixed prior to extraction to include the catnip, valerian root, black tea, thyme, mango, banana and slippery elm, in a ratio of about: 0.8-2.5 (catnip); 0.3-0.8 (valerian root); 0.1-0.5 (black tea); 0.5-1.5 (thyme); 1.0-3.0 (mango); 0.0-8 (banana); and 0.0-2.0 (slippery elm), respectively (based on ingredient dry weight). The ingredient mixture may be pre-mixed prior to extraction to include the catnip, valerian root, black tea, thyme, mango, banana and slippery elm in a ratio of about: 2 (catnip); 0.5 (valerian root); 0.25 (black tea); 1.0 (thyme); 2.0 (mango); 4.0 (banana); and 1.5 (slippery elm), respectively (based on ingredient dry weight).

The companion animal may be a feline.

The composition may be in a neat form.

The composition may further include a diluent. The diluent may be a water-based or an alcohol-based diluent.

According to another aspect of the invention, a method of reducing anxiety in a companion animal comprises exposing a companion animal to a composition as described above, wherein anxiety in said companion animal is reduced.

The companion animal may be exposed to the composition by inhalation of a diffused amount of the composition.

According to another aspect of the invention, a composition as described above is used for reducing anxiety in a companion animal.

According to another aspect of the invention, a method of manufacturing a composition for reducing anxiety in a companion animal comprises: (a) obtaining a pre-mixed ingredient mixture including catnip, valerian root, black tea, thyme, mango and, optionally, one or more of banana and slippery elm; and (b) extracting said ingredient mixture to yield a composition as described above useful for reducing anxiety in a companion animal.

The method may further comprise adding a natural preservative to said extract following extraction. The method may further comprise adding the lavender to said extract following extraction.

According to another aspect of the invention, a composition for reducing anxiety in a companion animal is manufactured by a process comprising: (a) obtaining a pre-mixed ingredient mixture including: catnip, valerian root, black tea, thyme, mango and, optionally, one or more of banana and slippery elm; and (b) extracting said ingredient mixture to yield a composition as described above for reducing anxiety in a companion animal.

According to another aspect of the invention, an anxiety treatment system for a companion animal, comprises: (a) a diffuser; and (b) a composition as described above, wherein said composition reduces anxiety in a companion animal inhaling an amount of the composition diffused by the diffuser.

The composition as described above may be in the form of a diffuser refill for use in an anxiety treatment system for a companion animal.

According to another aspect of the invention, an environmental enrichment article to treat anxiety in a companion animal, comprises: an environmental enrichment article; and a composition as described above. The article may be a toy, mat, platform, scratching post or bed.

According to another aspect of the invention, a laundry dryer sheet for reducing anxiety in a companion animal, comprises (a) a laundry dryer sheet; and (b) a composition as described above contained in said laundry sheet, wherein said composition reduces anxiety in a companion animal inhaling an amount of the composition that is present in an item that has been laundered with the laundry dryer sheet.

### DETAILED DESCRIPTION OF THE INVENTION

### I. In General

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

As used herein, the term “neat” refers to a liquid not diluted or mixed with any additional ingredient.

“Microwave-assisted extraction” (MAE), or simply “microwave extraction,” is an extraction technique that combines microwave and traditional solvent extraction. Application of microwaves for heating solvents and plant tissues in extraction process, which increases the kinetic energy of extraction, has a number of advantages, e.g., shorter extraction time, less solvent, higher extraction rate and lower cost, over traditional methods of extraction of natural products.

A “natural preservative” is a naturally-occurring substance that is added to products such as foods, pharmaceuticals, etc. to prevent decomposition by microbial growth or by undesirable chemical changes. Among the most effective natural preservatives are essential oils and various herbs such as rosemary, clove, thyme, cinnamon, tea tree, lavender, neem, and grape seed, which are more organism specific than their synthetic counterparts.

As used herein, the term “companion animal” shall refer to a pet or other domestic animal, including, but not limited to, a cat, dog, gerbil, hamster, guinea pig, ferret, rabbit, and bird. The term “feline” refers to a domestic cat or other member of the cat family.

### II. The Invention

The present invention is largely related to the field of companion animal care. More particularly, the present invention relates to a composition for reducing anxiety in a companion animal, particularly a feline. The invention further provides methods of manufacturing and using the composition, including methods of reducing anxiety in a companion animal by, for example, diffusion-based techniques.

Accordingly, in a first aspect, the present invention provides a composition for reducing anxiety in a companion animal, comprising an extract of an ingredient mixture including catnip, valerian root, black tea, thyme, mango and, optionally, one or more of banana and slippery elm, the composition further including lavender.

The inventive composition may further contain a natural preservative, such as, for example, citric acid, an essential oil, or an herb. In certain preferred embodiments, the natural preservative is a vinegar, more specifically apple cider vinegar.

The ingredient mixture may be extracted by steam distillation, microwave assisted extraction (MAE), liquid-liquid (solvent) extraction, ultrasound assisted extraction, or any other known extraction methods to yield the extract.

Inventive compositions are based on an ingredient mixture that is pre-mixed prior to extraction to include the catnip, valerian root, black tea, thyme, lavender (or added after extraction), mango, banana and slippery elm, in a ratio of about: 0.8-2.0 (catnip); 0.3-0.8 (valerian root); 0.1-0.5 (black tea); 0.5-1.5 (thyme); 0.001-0.003 (lavender); 1.0-3.0 (mango); 0.0-8 (banana); and 0.0-2.0 (slippery elm), respectively (based on ingredient dry weight). Optionally, the mixture further includes mango tea in a ratio of about 0.5-2 (mango tea). Certain preferred embodiments utilize an ingredient mixture that is pre-mixed prior to extraction to include the catnip, valerian root, black tea, thyme, lavender, mango, banana and slippery elm in a ratio of about: 2 (catnip); 0.5 (valerian root); 0.25 (black tea); 1.0 (thyme); 0.002 (lavender) (or added after extraction); 2.0 (mango); 4.0 (banana); and 1.5 (slippery elm), respectively (based on ingredient dry weight). If mango tea is added, it is prepared in a similar manner as the black tea.

Compositions of the invention may be provided in a neat form, or, alternatively, may further include a diluent. Suitable diluents include both water-based and alcohol-based diluent systems.

In a second aspect, the invention provides a method of reducing anxiety in a companion animal, comprising exposing a companion animal to a composition described and claimed herein, wherein anxiety in the companion animal is reduced. Delivery to the companion animal is accomplished by inhalation of a diffused amount of the composition by the respective animal.

In a third aspect, the present invention encompasses a method of manufacturing a composition for reducing anxiety in a companion animal, comprising: (a) obtaining a pre-mixed ingredient mixture including catnip, valerian root, black tea, thyme, mango and, optionally, one or more of banana and slippery elm; and (b) extracting the ingredient mixture to yield a composition described and claimed herein that is useful for reducing anxiety in a companion animal. The composition may further include lavender.

It can be appreciated that inventive compositions for reducing anxiety in a companion animal are manufactured by a process comprising: (a) obtaining a pre-mixed ingredient mixture including: catnip, valerian root, black tea, thyme, mango and, optionally, one or more of banana and slippery elm; and (b) extracting the ingredient mixture to yield a composition according to the present invention for reducing anxiety in a companion animal. The composition may further include lavender.

As can be appreciated, compositions according to the invention are useful in reducing anxiety in a companion animal, particularly a feline, in the context of a diffused odor or scent inhaled by the target feline. Accordingly, the inventive compositions may be incorporated in systems (e.g., a diffuser system or laundry sheet) and articles (e.g., a cat toy) to provide an anxiety reduction functionality to the respective system or article.

In a fourth aspect, an anxiety treatment system for a companion animal is provided. Such a system includes: (a) a diffuser; and (b) a composition according to the invention, wherein the composition reduces anxiety in a companion animal inhaling an amount of the composition diffused by the diffuser.

In a related embodiment, the compositions described and claimed herein may be provided in the form of a diffuser refill for use in an anxiety treatment system for a companion animal.

Yet another aspect of the invention is directed to an environmental enrichment article to treat anxiety in a companion animal. Such an article includes: an environmental enrichment article; and a composition according to the invention disposed in or on the article, wherein a companion animal is exposed to the beneficial effects of the composition by contact with the article. Specific embodiments of this inventive aspect are, for example, in the form of a toy, mat, platform, scratching post or animal bed.

In a similar aspect, the invention provides a laundry dryer sheet for reducing anxiety in a companion animal. Such an article includes (a) a laundry dryer sheet; and (b) a composition according to the invention contained in the laundry sheet, wherein the composition reduces anxiety in a companion animal inhaling an amount of the composition that is present in an item that has been laundered with the laundry dryer sheet.

Other objects, features and advantages of the present invention will become apparent after review of the specification, claims and any accompanying drawings. The detailed description and examples enhance the understanding of the invention, but are not intended to limit the scope of the appended claims.

### III. EXAMPLES

In this section, the inventors describe various materials, methods and results related to and supportive of the present invention.

Example 1. This example describes the preparation of an exemplary composition according to the invention useful for reducing anxiety in a companion animal, particularly a feline.

Ingredient List

Catnip (CN): 2 oz

Valerian Root (VR): 0.5 oz

Black Tea (BT): 0.25 oz

Thyme (T): 1.0 oz

Lavender (L): 0.002 oz (approx. 1-2 drops)

Banana (B): 1 whole, peeled fruit—approx. 4 oz.

Mango (M): 2 oz

Slippery Elm (SE): 1.5 oz

EITHER ¾ tbsp of Citric Acid (CA) OR 1 ounce of Apple Cider Vinegar (ACV).

The ingredients were obtained in dried forms and kept frozen prior to measuring and distillation. In some instances, the ingredients may be rehydrated prior to extraction with deionized water. It is understood that rehydration of the ingredients prior to measuring and distillation is optional. Banana and mango may undergo specific preparation steps and were initially-stored in a freezer and directly-added in non-dehydrated, natural form into the mixture. Dried forms of ingredients are widely available; exemplary commercial sources include Nuts.com, Frontier Co-op, Penzeys Spices, Walmart, Pick-n-Save, Amazon. Specific preparation, storage, pre-mixing and, optionally, rehydration procedures, are described in the subsequent paragraph.

CN, BT, VR, T may be separately-ground in a blender and then were weighed out to their appropriate weights listed above. It is understood that grinding of the ingredients prior to measurement and distillation is optional. CN was placed in a small Ziploc freezer bag as a 1.5 oz unit. VR, BT, T were placed in their individual weights into a freezer bag together. M was freezer bagged in 2 oz units and B was separately-frozen. SE was kept at room temperature. SE is the only dry ingredient not ground up and not frozen prior to being steam distilled. There is no set time that the ingredients were frozen; the ingredients were stored in the freezer until steam distilled. Immediately prior to distillation, the prepackaged units were placed in herb bags. SE was wrapped in cheese cloth. The ingredients were then microwaved at 500-1000 Watts for 1 minute and, subsequently, the ingredients were pre-mixed and rehydrated with deionized water prior to steam distillation.

The ingredients CN, BT, VR, T, M, B, SE were distilled together by purifying the mixture by vaporizing, then condensing the vapor by cooling the vapor, and collecting the resulting liquid into a 1 gallon jug. Next, ¾ tbsp. of CA or ACV were added to the 1 gallon jug. The resulting liquid is then separated to fill 4 oz. bottles. Lastly, 4 drops of L were added to each 4 oz. bottle.

Example 2. This example illustrates the anxiety reducing characteristics of a composition prepared according to the invention. More specifically, this example provides behavioral results related to reduction of anxiety in felines using the composition described in Example 1. Five domestic cats were exposed by diffusion to the composition and, by visual observation, all five cats exhibited reductions in their motor activities after exposure to the composition. More specifically, the five subjects immediately-exhibited behaviors indicative of reduced anxiety in felines upon inhaling the diffused composition. The composition was provided in close proximity to the cats as a single drop on the surface of a hard floor. The observed behaviors correlated with reduced anxiety in the animals and included drowsiness, sleep, intense smelling of the test area, and playful rolling.

As can be appreciated, the results described in the above examples support the utility of the composition described and claimed herein for reducing anxiety in a companion animal, particularly a feline. Other embodiments and uses of the invention will be apparent to those skilled in the art from consideration from the specification and practice of the invention disclosed herein. All references cited herein for any reason, including all journal citations and U.S./foreign patents and patent applications, if present, are specifically and entirely incorporated herein by reference. It is understood that the invention is not confined to the specific compositions, materials, methods, formulations, manufacturing conditions, etc., herein illustrated and described, but embraces such modified forms thereof as come within the scope of the following claims.

We claim:

1. A composition for reducing anxiety in a companion animal, comprising an extract of an ingredient mixture including catnip, valerian root, black tea, thyme, mango and, optionally, one or more of banana and slippery elm, the composition further comprising lavender.

2. The composition of claim 1, further comprising a natural preservative.

3. The composition of claim 2, wherein the natural preservative is citric acid, an essential oil, or an herb.

4. The composition of claim 2, wherein the natural preservative is a vinegar.

5. The composition of claim 1, wherein the ingredient mixture is extracted by steam distillation to yield the extract.

6. The composition of claim 1, wherein the ingredient mixture is extracted by microwave assisted extraction (MAE) to yield the extract.

7. The composition of claim 1, wherein the ingredient mixture is pre-mixed prior to extraction to include the catnip, valerian root, black tea, thyme, mango, banana and slippery elm, in a ratio of about: 0.8-2.5 (catnip); 0.3-0.8 (valerian root); 0.1-0.5 (black tea); 0.5-1.5 (thyme); 1.0-3.0 (mango); 0.0-8 (banana); and 0.0-2.0 (slippery elm), respectively (based on ingredient dry weight).

8. The composition of claim 7, wherein the ingredient mixture is pre-mixed prior to extraction to include the catnip, valerian root, black tea, thyme, mango, banana and slippery elm in a ratio of about: 2 (catnip); 0.5 (valerian root); 0.25 (black tea); 1.0 (thyme); 2.0 (mango); 4.0 (banana); and 1.5 (slippery elm), respectively (based on ingredient dry weight).

9. The composition of claim 1, wherein the companion animal is a feline.

10. The composition of claim 1, in a neat form.

11. The composition of claim 1, further including a diluent.

12. The composition of claim 11, wherein the diluent is a water-based or an alcohol-based diluent.

13. A method of reducing anxiety in a companion animal, comprising exposing a companion animal to a composition according to claim 1, wherein anxiety in said companion animal is reduced.

14. The method of claim 13, wherein the companion animal is exposed to the composition by inhalation of a diffused amount of the composition.

15. A composition according to claim 1 for use in reducing anxiety in a companion animal.

16. A method of manufacturing a composition for reducing anxiety in a companion animal, comprising: (a) obtaining a pre-mixed ingredient mixture including catnip, valerian root, black tea, thyme, mango and, optionally, one or more of banana and slippery elm; and (b) extracting said ingredient mixture to yield a composition according to claim 1 useful for reducing anxiety in a companion animal.

17. The method of claim 16, further comprising adding a natural preservative to said extract following extraction.

18. The method of claim 17, further comprising adding the lavender to said extract following extraction.

19. A composition for reducing anxiety in a companion animal manufactured by a process comprising: (a) obtaining a pre-mixed ingredient mixture including: catnip, valerian root, black tea, thyme, mango and, optionally, one or more of banana and slippery elm; and (b) extracting said ingredient mixture to yield a composition according to claim 1 for reducing anxiety in a companion animal.

20. An anxiety treatment system for a companion animal, comprising: (a) a diffuser; and (b) a composition according to claim 1, wherein said composition reduces anxiety in a companion animal inhaling an amount of the composition diffused by the diffuser.

21. The composition of claim 1 in the form of a diffuser refill for use in an anxiety treatment system for a companion animal.

22. An environmental enrichment article to treat anxiety in a companion animal, comprising: an environmental enrichment article; and a composition according to claim 1.

23. The environmental enrichment article of claim 21, wherein said article is a toy, mat, platform, scratching post or bed.

24. A laundry dryer sheet for reducing anxiety in a companion animal, comprising (a) a laundry dryer sheet; and (b) a composition according to claim 1 contained in said laundry sheet, wherein said composition reduces anxiety in a companion animal inhaling an amount of the composition that is present in an item that has been laundered with the laundry dryer sheet.
